(19) United States
(12) Patent Application Publication
(10) Pub. No.: US 2004/0151788 A1
(43) Pub. Date: Aug. 5, 2004

(54) Method for increasing bone mineral density

(76) Inventors: Oscar S. Gluck, Phoenix, AZ (US); Michael Joseph Maricic, Tucson, AZ (US)

Correspondence Address: WEISS & MOY PC, 4204 NORTH BROWN AVENUE, SCOTTSDALE, AZ, 85251, US

(21) Appl. No.: 10/359,515

(22) Filed: Feb. 5, 2003

(51) Int. Cl. A61K 35/78; A61K 31/66

(52) U.S. Cl. 424/729; 514/102

(57) ABSTRACT

A method for increasing bone mineral density, that includes the substantially daily consumption of at least one cup of tea for a period of years. Preferably, such consumption continues for at least about six years. Where utilized as a treatment for osteoporosis rather than as a preventative measure, it is preferred that the tea consumption be accompanied by treatment with osteoporosis-treating pharmaceutical agents, such as anti-resorptives or bone forming agents. The method should include measurements of bone mineral density, preferably occurring on a periodic basis.

### FIELD OF THE INVENTION

This invention relates generally to methods for increasing bone mineral density and, more particularly, to a method for increasing bone mineral density through the consumption of tea in combination with anti-resorptive and/or bone forming pharmaceutical agents.

### BACKGROUND OF THE INVENTION

Osteoporosis is a skeletal disorder that is characterized by reduced bone strength that creates an increased risk of fractures, height loss, hunched backs, and considerable pain. There are three main causes of osteoporosis: (a) an increased rate of bone loss at menopause in women or as men and women age, as a result of decreased hormone production (estrogen in women and testosterone in men); (b) less than optimal bone growth during childhood and adolescence; and (c) bone loss that is secondary to disease conditions, eating disorders, or certain medications and medical treatments. More than one of these factors may contribute to a case of osteoporosis. Other risk factors for osteoporosis include a short body build, weight, smoking, calcium deficiency or malabsorption, vitamin D deficiency, lack of exercise, and ingestion of certain drugs Osteoporosis causes more than 250,000 hip fractures a year. Complications from osteoporosis are the 12th leading cause of death in the United States.

Bone strength is a function both of bone mineral density (“BMD”) and bone quality. Bone goes through a constant process of renewal. Remodelling refers to the continual replacement of old bone tissue by new bone tissue. There are two aspects to bone remodelling—resorption, the destruction of bone matrix, caused by osteoclasts “eating up” old bone, and bone formation, whereby osteoblast cells replace old bone with new bone. In osteoporosis, bone resorption outpaces bone formation.

The onset of osteoporosis can be prevented or treated through a combination of an adequate calcium intake and exercise (particularly weight-bearing exercise). For post-menopausal women, estrogen replacement therapy, consisting of low does of estrogen and sometimes progesterone, may also be employed. Another treatment involves the use of agents that inhibit bone resorption. Alendronate sodium is one example of an inhibitor of osteoclast-mediated bone resorption. Efforts are also underway to develop bone formation agents; i.e., agents that will promote new bone growth.

Tea is a beverage that enjoys wide popularity. After water, tea is the most common drink consumed regularly by most adults in the world. There are three types of tea: (a) green (nonfermented); (b) oolong (partially fermented); and (c) black (fermented). Tea is a rich source of flouride, and also contains high concentrations of caffeine, flavonoids and phytoestrogen.

A need exists for improved methods of combatting bone fracture risk accompanied by low BMD. Such methods should preferably be utilizable prophylactically, as a guard against the development of low BMD. Alternatively, they may be employed as a treatment for patients that already suffer from low BMD.

The present invention concerns a method for increasing BMD, based on a program of regular (preferably at least daily) tea consumption, and preferably wherein the tea consumption is in combination with the ingestion of anti-resorptives and/or bone forming agents.

### SUMMARY OF THE INVENTION

It is an object of the present invention to provide a method for providing increased BMD.

It is a further object of the present invention to increase BMD through a program of regular and sustained tea ingestion.

It is a still further object of the present invention to increase BMD through a program of regular and sustained tea ingestion, in combination with the ingestion of one or more anti-resorptive agents.

It is a yet further object of the present invention to increase BMD through a program of regular and sustained tea ingestion, in combination with the ingestion of one or more bone forming agents.

### BRIEF DESCRIPTION OF THE PREFERRED EMBODIMENTS

In accordance with one embodiment of the present invention, a method for increasing bone mineral density is disclosed. The method comprises the steps of: as a means for one of preventing onset of osteoporosis and treating osteoporosis, a patient consuming tea on a substantially daily basis; wherein the consumption of tea continues for a period of years; and measuring the patient's bone mineral density.

In accordance with another embodiment of the present invention, a method for increasing bone mineral density is disclosed. The method comprises the steps of: as a means for one of preventing onset of osteoporosis and treating osteoporosis, a patient consuming tea on a substantially daily basis; wherein at least one cup of the tea is consumed substantially daily; wherein the consumption of tea continues for at least six years; and measuring the patient's bone mineral density.

In accordance with another embodiment of the present invention, a method for increasing bone mineral density is disclosed. The method comprises the steps of: as a means for one of preventing onset of osteoporosis and treating osteoporosis, a patient consuming tea on a substantially daily basis; wherein at least one cup of the tea is consumed substantially daily; wherein the consumption of tea continues for at least six years; ingesting an anti-resorptive pharmaceutical agent; wherein the anti-resorptive pharmaceutical agent is alendronate sodium; and measuring the patient's bone mineral density.

The foregoing and other objects, features, and advantages of the invention will be apparent from the following, more particular, description of the preferred embodiments of the invention, as illustrated in the accompanying drawings.

### DETAILED DESCRIPTION OF THE PREFERRED EMBODIMENTS

The method of the present invention concerns the ingestion of tea, preferably in order to prevent the onset of osteoporosis and as an osteoporosis treatment. In one embodiment, tea may be ingested without the combination of any pharmaceutical agents for the treatment of osteoporosis—such as anti-resorptive or bone forming agents. This method would particularly be preferred where tea ingestion is utilized to prevent osteoporosis, or otherwise where the consumption of osteoporosis-treating pharmaceutical agents would not be appropriate or desirable.

In a preferred embodiment, tea is ingested in combination with osteoporosis-treating pharmaceutical agents. These could include, for example, alendronate sodium. These different embodiments will now be described in more detail.

The basic steps in the method of the present invention include the long term, regular, ingestion of tea by a patient. The tea can be green, oolong, or black. Such ingestion should preferably be approximately daily, with at least one cup of tea being consumed. To have significant beneficial affect, substantially daily ingestion should occur over a period of years. Preferably, such ingestion should continue at least for a period of about six years. It should be clearly understood that specific components of tea that are found to be the active ingredients responsible for bone density increase could be isolated or extracted and administered in the same method described herein for the ingestion of tea as a whole.

In combination with tea ingestion, and where the method of the present invention is utilized in the treatment of osteoporosis, it would be preferred to combine tea ingestion with appropriate dosages of osteoporosis-treating agents. This would include anti-resorptive and/or bone growth promoting agents. As an example, it would be preferred to combine tea consumption with the ingestion of appropriate dosage levels of the anti-resorptive alendronate sodium, although it should be clearly understood that substantial benefit could be derived from other anti-resorptive agents.

The long term ingestion of tea should lead to increases in BMD. This may occur in some or all of the total body, lumbar spine, hip, neck and Ward's triangle (a triangular area within the femoral neck on which the trabeculae are normally thin and loosely arranged) regions. The increase in BMD is thought to be attributable to a number of things. First, fluoride intake has been shown to alleviate osteoporotic progression, and tea leaves are a rich source of protein. Second, tea is a source of isoflavonoids, and these have a weak estrogenic effect. Given that tea contains over 4,000 chemical compounds, it is possible that others of these may also play a role in increasing BMD.

It is thought that the combination of tea consumption and the ingestion of anti-resorptives and/or bone forming agents will cause a synergistic effect, increasing the effectiveness of both if consumed alone.

It would be preferred, in combination with one or more of the embodiments disclosed herein, to subject the patient to periodic measurement of BMD. Such measurement can be undertaken using dual-energy X-ray absorptiometry, such as with the Hologic ADR-1000 sold by Hologic, Inc. The results of such BMD measurements can be used to measure a patient's progress, and to inform decisions concerning any change or adjustment in the method—such as the addition of a pharmaceutical agent, the change to a new agent, an increase in daily tea consumption, or the like.

While the invention has been particularly shown and described with reference to preferred embodiments thereof, it will be understood by those skilled in the art that the foregoing and other changes in form and details may be made therein without departing from the spirit and scope of the invention.

I claim:

1. A method for increasing bone mineral density, comprising the steps of:
as a means for one of preventing onset of osteoporosis and treating osteoporosis, a patient consuming tea on a substantially daily basis;
wherein said consumption of tea continues for a period of years; and
measuring said patient's bone mineral density.

2. The method of claim 1 wherein said tea is green.

3. The method of claim 1 wherein said tea is oolong.

4. The method of claim 1 wherein said tea is black.

5. The method of claim 1 wherein at least one cup of said tea is consumed substantially daily.

6. The method of claim 1 wherein said substantially daily consumption of tea continues for at least six years.

7. The method of claim 1 further comprising the step of ingesting an anti-resorptive pharmaceutical agent.

8. The method of claim 7 wherein said anti-resorptive pharmaceutical agent is alendronate sodium.

9. The method of claim 1 further comprising the step of ingesting a bone forming agent.

10. The method of claim 7 further comprising the step of ingesting a bone forming agent.

11. The method of claim 1 wherein said measuring occurs yearly.

12. A method for increasing bone mineral density, comprising the steps of:
as a means for one of preventing onset of osteoporosis and treating osteoporosis, a patient consuming tea on a substantially daily basis;
wherein at least one cup of said tea is consumed substantially daily;
wherein said consumption of tea continues for at least six years; and
measuring said patient's bone mineral density.

13. The method of claim 12 further comprising the step of ingesting an anti-resorptive pharmaceutical agent.

14. The method of claim 13 wherein said anti-resorptive pharmaceutical agent is alendronate sodium.

15. The method of claim 12 further comprising the step of ingesting a bone forming agent.

16. The method of claim 13 further comprising the step of ingesting a bone forming agent.

17. A method for increasing bone mineral density, comprising the steps of:
as a means for one of preventing onset of osteoporosis and treating osteoporosis, a patient consuming tea on a substantially daily basis;
wherein at least one cup of said tea is consumed substantially daily;
wherein said consumption of tea continues for at least six years;
ingesting an anti-resorptive pharmaceutical agent;
wherein said anti-resorptive pharmaceutical agent is alendronate sodium; and
measuring said patient's bone mineral density.
